(19) United States
(12) Patent Application Publication
(10) Pub. No.: US 2024/0160208 A1
(43) Pub. Date: May 16, 2024

(54) MIXED MODE MOBILE SERVICE ROBOT WITH MANUAL AND AUTONOMOUS MANEUVERABILITY

(71) Applicant: Loop Robots, Rijswijk (NL)

(72) Inventors: Per Slycke, Rijswijk (NL); Aswin Chandarr, Rijswijk (NL)

(21) Appl. No.: 17/988,555

(22) Filed: Nov. 16, 2022

(51) Int. Cl. G05D 1/00; A61L 2/10; A61L 2/24

(52) U.S. Cl. CPC G05D 1/0088; A61L 2/10; A61L 2/24; A61L 2202/11; A61L 2202/14; A61L 2202/16

(57) ABSTRACT

A mixed mode robot is provided having an autonomous mode, wherein the robot moves autonomously, and a manual mode, wherein the robot is passive and allows manual manipulation by a user. The mixed mode robot is wheeled and in an embodiment is powered by one or more direct drive motor while in the autonomous mode. One or more handholds are adapted for use by a user to move the robot when the robot is in the manual mode. A processor associated with the robot places the robot in a selected one of the autonomous mode and the manual mode, such that the robot is easily moved by a user when in the manual mode.

### TECHNICAL FIELD

The present disclosure is related generally to mobile service robots and, more particularly, to a mobile service robot having both manual and autonomous maneuverability for wide application across industries.

### BACKGROUND

While robots have been used often to save labor and associated costs, wide-scale commercial usage of autonomous mobile robots is currently limited to the domains of warehousing and industrial factory floors and other structured environments specifically designed to facilitate the use of mobile robots. One notable reason for this limitation in the inventors' view is that to achieve the required utility, the robots need full autonomy which is difficult and which indeed is generally economically unfeasible given the limited utility to be replaced.

In greater particularity, technology has reached a stage where robots can reliably perform many time-consuming, difficult and/or dangerous tasks for humans. However, it remains difficult for robots to perform many simple tasks that can be done by easily by a human. For example, in a restaurant setting, a logistics robot might move from a kitchen corner to a table simply and reliably. However, positioning itself in a useful manner next to the chef or diner is a much more difficult task for a robot to reliably perform.

Similarly, for a smart mobile disinfection robot with UVC functionality, scanning a room and delivering the right amount of UVC dosage in all regions of the room is achievable, while this task would be difficult and hazardous for a human. However, moving quickly from room to room in a large and busy hospital environment, going over floor thresholds, and performing other simple acts of navigation and maneuvering in a highly active and dynamic environment such as a hospital is a challenge for a robot. In a warehouse setting, a robot is able to move across vast spaces quickly and accurately, but positioning itself accurately next to a loading rack, packing counter or other variably-positioned element becomes a challenge.

Before proceeding to the remainder of this disclosure, it should be appreciated that the disclosure may address some of the shortcomings listed or implicit in this Background section. However, any such benefit is not a limitation on the scope of the disclosed principles, or of the attached claims, except to the extent expressly noted in the claims.

Additionally, the discussion of technology in this Background section is reflective of the inventors' own observations, considerations, and thoughts, and is in no way intended to be, to accurately catalog, or to comprehensively summarize any prior art reference or practice. As such, the inventors expressly disclaim this section as admitted or assumed prior art. Moreover, the identification or implication herein of one or more desirable but unfollowed courses of action reflects the inventors' own observations and ideas, and should not be assumed to indicate an art-recognized desirability.

### BRIEF DESCRIPTION OF THE SEVERAL VIEWS OF THE DRAWINGS

While the appended claims set forth the features of the present techniques with particularity, these techniques, together with their objects and advantages, may be best understood from the following detailed description taken in conjunction with the accompanying drawings of which:

FIG. 1 is a simplified line drawing of an example disinfection robot within which embodiments of the disclosed principles may be implemented;

FIG. 2 is a simplified line drawing of the example disinfection robot of FIG. 1, wherein the disinfection robot is shown in perspective view, facing slightly toward the viewer;

FIG. 3 is a simplified internal side view of the robot base showing operational modules in accordance with an embodiment of the disclosed principles;

FIG. 4 is a flowchart showing an example process of mixed mode operation in a warehouse environment in accordance with an embodiment of the disclosed principles;

FIG. 5 is a flowchart showing a generalized process for mode management in accordance with an embodiment of the disclosed principles; and

FIG. 6 is a collection of decision tables showing a sensor-based switching schema for moving between autonomous and manual modes in accordance with an embodiment of the disclosed principles.

### DETAILED DESCRIPTION

Before presenting a detailed discussion of embodiments of the disclosed principles, an overview of certain embodiments is given to aid the reader in understanding the later discussion. As noted above, currently available autonomous mobile robots may have substantial capabilities, but they lack the ability to allow or facilitate other tasks at all.

In an embodiment of the disclosed principles, a robot design is employed having both manual and autonomous maneuverability for application in environments that combine active and dynamic or variable environments like hospital hallways with more predictable settings such as large open spaces. Considering an example mentioned above, it is difficult for an autonomous warehouse robot to position itself next to a loading rack or packing counter. However, it would be a simple matter for a human operator to complement the robot's abilities to accomplish this task.

Consider the example of a restaurant environment; it is easy for the chef or diner to pull a cart to the right position to pick up or place down the food. In a hospital environment, a staff member such as a cleaner can move a cart across crowded spaces, over thresholds, and elevators and bring it to the room which is to be cleaned, whereupon the autonomous functions of the robot may then take over.

The ability of mobile robots to switch modes between “smart fully autonomous agents with high reliability” and “very trivial, simple passive cart-like devices” is the key to bringing widespread commercial and cost-effective adoption of robots in various domains where they have great utility. There are a number of factors preventing current autonomous robots from being manually operated by personnel, and indeed, currently no autonomous robots are known to have handles designed for easy and ergonomic manual manipulation.

The present disclosure provides a mixed mode mobile robot having embedded mechanisms that allow them to operate in these two drastically different modes and provide a seamless transition between the modes. Such robots are not designed to replace humans but to act as high-performance tools that will aid the effectiveness of human operators. These mixed mode mobile robots interact with humans, operate around humans and benefit from being easily manipulated by humans in an ergonomic fashion.

The manual mode of the disclosed robot allows a user to manually move the robot from one location to another easily without requiring using a remote control or prior mapping of the space the robot is moving in, allowing intuitive manual positioning of the robot in tight spaces, close to objects and humans. The transition between modes (manual or autonomous) can be triggered via any of a number of mechanisms including, for example, a touch control in the handles or other surfaces intended for human operators, one or more push buttons, touch detection (via sensors such as capacitive sensors), or in an entirely automatic manner. An example of the latter is a torque sensor on one or more wheels coupled with an inertial sensor.

More importantly, the manual mode of the disclosed robot leverages physical features that allow the robot to be manually manipulated like a simple cart, without needing any active force sensing and motion control unlike existing devices. As such, a new class of robots is defined, i.e., mixed mode mobile robots. These wheeled mobile robots are highly specialized tools for supporting human operators while also explicitly designed to allow manual manipulation. Such robots operate as highly advanced autonomous systems when performing a particular specialized task suitable for automation, but when not doing such a task, the device acts as a passive mobile cart that can be easily moved around and manipulated by human operators without using force sensing, remote controls or hand controls to operate motors or other motion aides. The device switches between autonomous and passive modes by simple touch of a human operator, by voice command, by proximity sensing, or otherwise.

With hub motors, which include a high resolution encoder and standard hall sensors, powering the drive wheels in autonomous mode, the robot becomes easily and ergonomically pushable when the hub motors are depowered for manual mode. In an embodiment, each high resolution encoder provides at least 3200 counts per rotation.

The robot disclosed herein may have one or more handholds adapted for use by a user to push the robot when the robot is in a manual mode. One or more hand grips may be located on the robot at or above the user's shoulder level and one or more mid hand grips may be located on the robot below the user's shoulder level for ergonomic manual manipulation, including pushing and pulling.

The wheels may be of any size, but in an embodiment the wheels are at least 10 cm in diameter, and the wheels having direct drive hub motors have diameters of at least 15 cm in a further embodiment.

One or more tilt support plates are provided, each tilt support plate being a small angled extension placed at the bottom of the robot and facing a user pushing the robot. In an embodiment, the user can press them with their foot while pulling the robot towards them to tilt the robot. The one or more tilt support plates may be user-removable and/or user-foldable and are preferably dimensioned to prevent toppling of the robot.

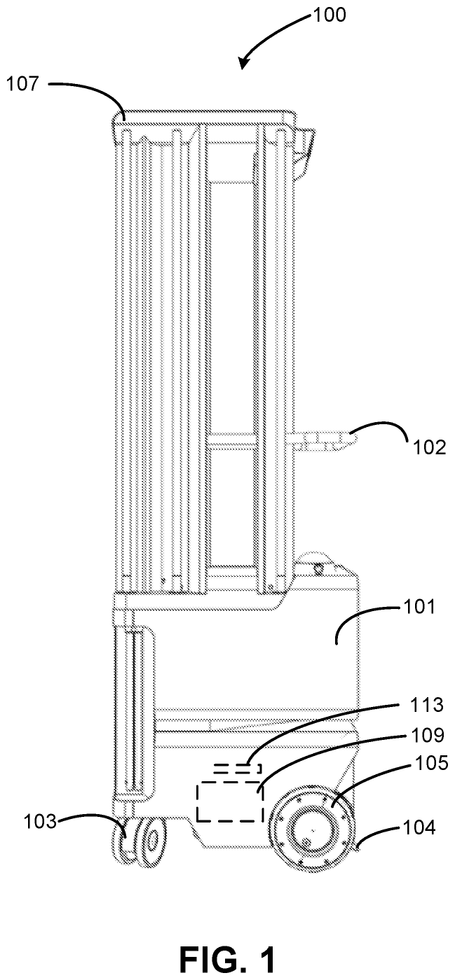

With this overview in mind, and turning now to a more detailed discussion in conjunction with the attached figures, the techniques of the present disclosure are illustrated as being implemented in or via a suitable device environment. Thus, for example, FIG. 1 illustrates an example disinfection robot which may embody one or more aspects of the disclosed principles. It should be noted that the features of the robot may be adapted for its intended usage environment. So while the illustrated example includes UVC lights, a logistics robot, for example, may include arms or manipulators for lifting, moving and retaining objects.

In the illustrated embodiment, the disinfection robot 100 includes a base 101, which is supported, driven and steered by wheels 103, 105. A handle 102 allows a human operator to push and steer the robot in manual mode, and a stop 104 prevents the robot from tipping backward when encountering ridges or bumps during manual operation. Thus, while a user may deliberately tilt the robot further depending upon the user's strength, the tip prevention stop 104 prevents inadvertent over-center tipping by maintaining a position wherein the robot center of mass remains along a vertical axis that passes between wheel centers.

In the illustrated example, the robot 100 includes a light tower 107, which holds one or more UVC emitting lights. The illustrated tower 107 is merely to indicate a region wherein lights may be mounted and is not intended to illustrate a particular shape or number of lights. It will be appreciated that the disinfection robot 100 may include UVC emitting lights in other positions in addition to or instead of the light tower 107.

The disinfection robot 100 includes a power source 109 such as a battery, for powering the navigation of the disinfection robot 100 as well as the one or more UVC emitting lights. A wall charging station may be provided for periodically recharging the power source 109 or the robot 100 can be directly connected to mains power outlets by means of a cable with a plug. The disinfection robot 100 further includes a processor system 113 for executing the robot-based activities discussed herein. A collar may be used to enable physical and electrical separation of the light tower 107 from the main body of the robot 101. Necessary or desirable peripheral systems such as sensors, LIDAR sensors, cameras, motion tracking sensors, computer memory, latches, switches, antennas, communications facilities and so on are also included in the disinfection robot 100 but are omitted from the figure for clarity.

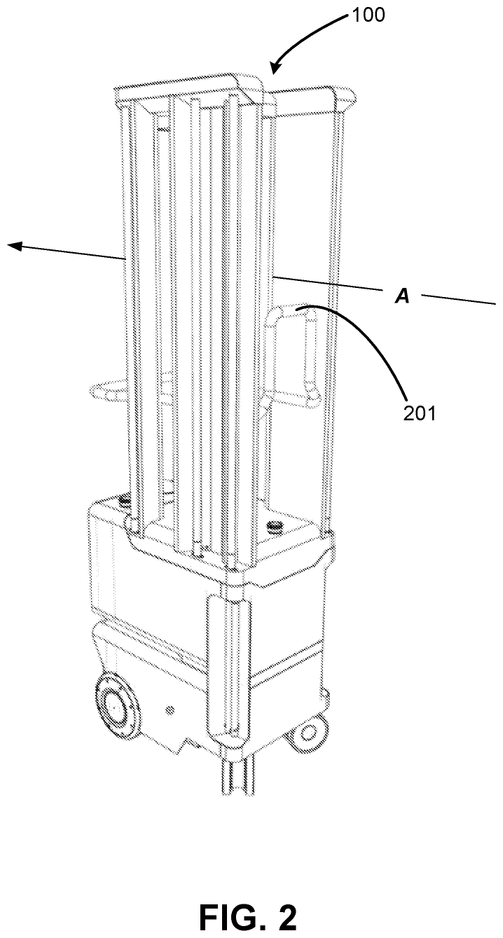

Turning to FIG. 2, the disinfection robot 100 is shown in perspective view, facing slightly toward the viewer. In this view, the forward-facing handle 201 (102) is shown in better detail, and may be used for manually manipulating the robot 101 from the front while the robot is in the manual mode. Also apparent in this view is the fact that the tower 107 is see-through. For example, a user behind the robot 100 is able to see along sight line A through the tower 107, to avoid unintentionally pushing the robot 100 into a wall, person, piece of furniture or other object while maintaining an ergonomic upright position while manually pushing or pulling the robot.

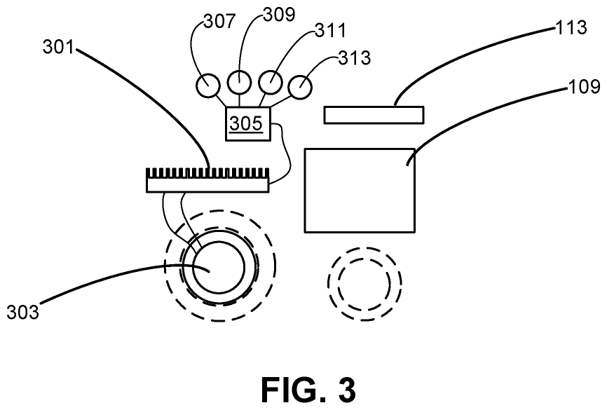

FIG. 3 is a simplified internal side view of the base 101 showing operational modules in greater detail. The power source 109 and processor system 113 as described above are shown here, as is a motor controller 301 and the right-side motor 303. It will be appreciated that the right-side motor 303 is located and configured to drive the right rear wheel, while a left-side motor (not shown in this view) is located and configured to drive the left rear wheel. In this way, the robot may navigate while in autonomous mode.

The right and left-side motors are direct drive; that is, they drive their respective wheels without the aid of intervening reduction gearing. Because of this, the rear wheels become manually rollable with ease when the motors are depowered, i.e., when the robot is placed in manual mode. In either mode, the front wheels may caster to allow operator steering in manual mode and differential drive steering in autonomous mode.

The illustrated base 101 also includes a mode switch sensor 305, linked to the motor controller 301 and receiving input from a proximity sensor 307, and touch sensor 309, a torque sensor 311 and an inertial measurement unit (IMU) 313. As will be discussed in greater detail later herein, any or all of the proximity sensor 307, touch sensor 309, torque sensor 311 and IMU 313 may be used to trigger a transition from the autonomous node to the manual mode.

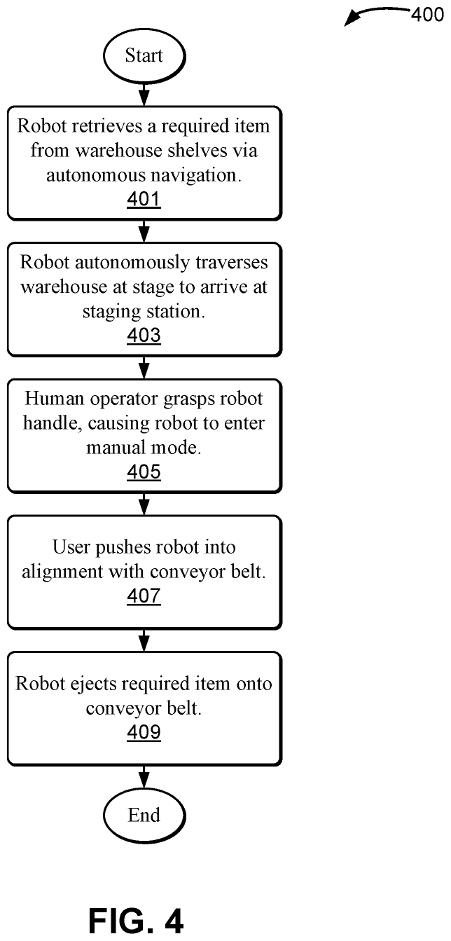

Although the operational scenarios of the described mixed modal robot are vast, FIG. 4 shows a simple example of mixed mode operation in a warehouse environment using an embodiment of the robot described herein. At stage 401 of the illustrated process 400, the robot retrieves a required item from warehouse shelves via autonomous navigation. Having retrieved the required item, the robot traverses the warehouse space at stage 403 to arrive at a staging station. A human operator grasps the robot handle at stage 405, causing the robot to switch into manual mode. The user then pushes the robot into alignment with a conveyor belt at stage 407, and at stage 409, the robot ejects, e.g., pursuant to a user touch prompt or otherwise, the required item onto the conveyor belt.

As noted in passing earlier in this specification, the mixed mode robot transitions from a fully autonomous mode to a passive pushable cart-like mode. In a preferred embodiment, the transition between these modes is executed in a manner that is trivial or even transparent for the user. A number of aspects that lend themselves to manual operation regardless of the switching mechanisms are also of interest, including pushability, the presence of handlebars, direct drive motors to reduce rolling resistance when unpowered, stability, and tilt support or tip-over prevention mechanisms. When the mode switch and the usability within the manual mode are assured, the issue of guidance is still important, and in an embodiment, this is assured by the see-through nature of the tower as shown in FIG. 2. The foregoing combine to provide ergonomic usability for the user.

Of course, the size and weight of the mixed mode robot should also be within limits that allow user manipulation. For example, robots should be no taller than necessary, e.g., six (6) feet tall, and should not be prohibitively massive, e.g., greater than about 200 kg.

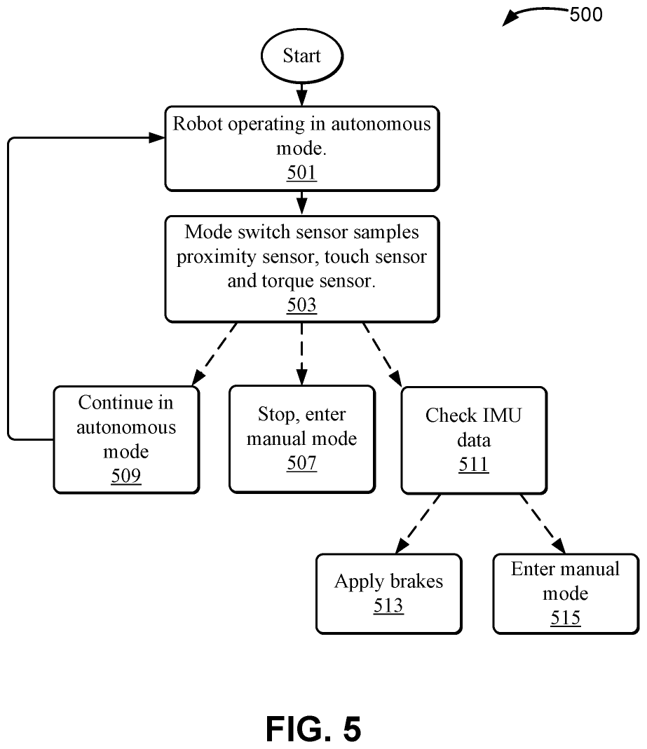

With the example of FIG. 4 showing operation of the system in a specific scenario, we turn to a more generalized process FIG. 5 another flow chart, more complicated due to its wider applicability. In this embodiment, the autonomous-to-manual mode switch is triggered by any of proximity, touch or a combination of torque and orientation.

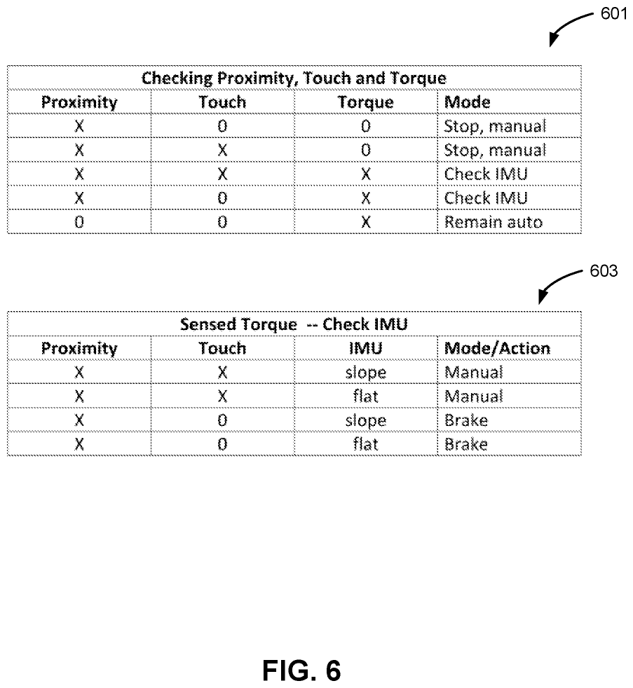

At stage 501 of the illustrated process 500, the robot is in the autonomous mode, and is executing a task. The mode switch sensor 305 of the robot samples the associated proximity sensor, touch sensor and torque sensor at stage 503, and at stage 505 of the process 500 executes the operations table 601 of FIG. 6, selectively stopping and entering the manual mode (stage 507), continuing operation in the autonomous mode (stage 509) or checking the IMU (stage 511).

If the process 500 has flowed to stage 511 to check the IMU, it executes operations in accordance with operations table 603 of FIG. 6, and either brakes (stage 513) or enters manual mode (stage 515).

As can be seen, the operations tables 601, 603, in conjunction with the robot's sensor suite, cause the robot to enter the manual mode in the event that the user is present and touching the robot, e.g., at a touch sensor on the handle(s), to brake if torque is experienced while a user is present but not touching the robot, and to otherwise ensure user safety while also ensuring that the switch from autonomous operation to manual operation is seamless to the user.

Given the foregoing, it can be seen that the applicability of the described robots goes far beyond disinfection, restaurant service or warehouse service. Depending upon the accessories included on the robot, it may serve any other function, including but not limited to security and patrol, inspection, transport and delivery, and all while either indoor or outdoor.

While the example shows the user switching the robot into manual mode via actions detected by a sensor suite, any suitable switching mechanism may be used, e.g., a user activation of a button or switch or automatic switching upon detecting a predetermined location, signal presence or object.

As noted above, the manual manipulation of the robot is by simply pushing and steering, not via control inputs such as levers, sliders or steering wheels for throttle, brakes, steering etc. That is to say, the described robot is not a user-operated force extender such as a bulldozer or forklift, but is a simple push cart when in manual mode, allowing for maximum flexibility and intuitive and trivial operation by untrained personnel, while maintaining a maximum level of safety.

It will be appreciated that various systems and processes have been disclosed herein. However, in view of the many possible embodiments to which the principles of the present disclosure may be applied, it should be recognized that the embodiments described herein with respect to the drawing figures are meant to be illustrative only and should not be taken as limiting the scope of the claims. Therefore, the techniques as described herein contemplate all such embodiments as may come within the scope of the following claims and equivalents thereof.

We claim:

1. A mixed mode robot having an autonomous mode, wherein the robot moves autonomously, and a manual mode, wherein the robot is passive and allows manual manipulation by a user, the mixed mode robot comprising:
a robot base having a plurality of wheels associated therewith, and with each of at least two wheels having associated therewith a direct drive motor to drive the robot when in the autonomous mode;
one or more handholds connected to the robot base and adapted for use by a user to move the robot when the robot is in the manual mode; and
a processor configured to place the robot in a selected one of the autonomous mode and the manual mode, to navigate the robot via the direct drive motors in the autonomous mode, and to depower the direct drive motors in the manual mode to allow the user to easily move the robot.

2. The mixed mode robot in accordance with claim 1, wherein the robot is a UVC site disinfection robot further comprising one or more UVC lights for site disinfection.

3. The mixed mode robot in accordance with claim 2, wherein the one or more UVC lights for site disinfection are supported in a tower connected to the robot base.

4. The mixed mode robot in accordance with claim 3, wherein the tower is connected to the robot base in a user-removable manner.

5. The robot in accordance with claim 1, wherein the processor is further configured to place the robot in a selected one of the autonomous mode and the manual mode based on a sensor suite comprising at least one of a proximity sensor, a touch sensor, a wheel torque sensor and an inertial measurement unit (IMU).

6. The robot in accordance with claim 5, wherein placing the robot in a selected one of the autonomous mode and the manual mode comprises determining based on data from the sensor suite whether the user is attempting to manually move the robot, and placing the robot in the manual mode if it is determined that the user is trying to manually move the robot.

7. The robot in accordance with claim 1, further comprising a user interface, and wherein the processor is further configured to place the robot in a selected one of the autonomous mode and the manual mode based on a user action relative to the user interface.

8. The robot in accordance with claim 7, wherein the user interface is one of a manual interface and a graphical user interface (GUI).

9. The robot in accordance with claim 1, wherein the mass of the robot is less than 200 kg.

10. The robot in accordance with claim 1, further comprising at least one projection on the base to prevent over-center tipping of the robot while being moved by the user or actuated by the processor autonomously.

11. The robot in accordance with claim 10, wherein the at least one projection is configured such that the user can press the projection with their foot while pulling the robot towards them via the handle to tilt the robot.

12. The robot in accordance with claim 10, wherein the at least one projection is foldable or removable by the user.

13. The robot in accordance with claim 1, wherein the robot is configured to allow a line of sight from the user through the robot from front to back while the user is moving the robot in manual mode.

14. A mixed mode UVC site disinfection robot having an autonomous mode, wherein the robot moves autonomously, and a manual mode, wherein the robot is passive and allows manual manipulation by a user, the mixed mode robot comprising:
a robot base having a plurality of wheels associated therewith, and with each of at least two wheels having associated therewith a direct drive motor to drive the robot when in the autonomous mode;
one or more UVC lights attached to the robot base for site disinfection;
one or more handholds connected to the robot base and adapted for use by a user to move the robot when the robot is in the manual mode; and
a processor configured to place the robot in a selected one of the autonomous mode and the manual mode, to navigate the robot via the direct drive motors in the autonomous mode, and to depower the direct drive motors in the manual mode to allow the user to easily move the robot.

15. The mixed mode robot in accordance with claim 14, wherein the one or more UVC lights for site disinfection are supported in a removable tower connected to the robot base.

16. The robot in accordance with claim 14, wherein the processor is further configured to place the robot in a selected one of the autonomous mode and the manual mode based on a sensor suite comprising at least one of a proximity sensor, a touch sensor, a wheel torque sensor and an inertial measurement unit (IMU).

17. The robot in accordance with claim 16, wherein placing the robot in a selected one of the autonomous mode and the manual mode comprises determining based on data from the sensor suite whether the user is attempting to manually move the robot, and placing the robot in the manual mode if it is determined that the user is trying to manually move the robot.

18. The robot in accordance with claim 14, further comprising a user interface, and wherein the processor is further configured to place the robot in a selected one of the autonomous mode and the manual mode based on a user action relative to the user interface, wherein the user interface is one of a manual interface and a graphical user interface (GUI).

19. The robot in accordance with claim 14, wherein the mass of the robot is less than 200 kg.

20. The robot in accordance with claim 14, further comprising at least one projection on the base to prevent over-center tipping of the robot while being moved by the user or actuated by the processor autonomously.

21. The robot in accordance with claim 20, wherein the at least one projection is configured such that the user can press the projection with their foot while pulling the robot towards them via the handle to tilt the robot.

22. The robot in accordance with claim 20, wherein the at least one projection is foldable or removable by the user.

23. The robot in accordance with claim 14, wherein the robot is configured to allow a line of sight from the user through the robot from front to back while the user is moving the robot in manual mode.
